(19) United States
(12) Patent Application Publication
(10) Pub. No.: US 2022/0362219 A1
(43) Pub. Date: Nov. 17, 2022

(54) RETINA REGENERATION THROUGH EPIGENETICS MANIPULATION

(71) Applicant: Baylor College of Medicine, Houston, TX (US)

(72) Inventor: Rui Chen, Houston, TX (US)

(21) Appl. No.: 17/757,058

(22) PCT Filed: Dec. 11, 2020
(86) PCT No.: PCT/US20/64543

§ 371 Date: Jun. 8, 2022

Related U.S. Application Data
(60) Provisional application No. 62/948,254, filed on Dec. 14, 2019.

(51) Int. Cl. A61K 31/423; A61K 31/4155; A61K 31/122; A61K 31/60; A61K 31/426; A61K 31/12; A61K 31/365; A61K 31/519; A61K 31/437; A61K 31/515; A61P 27/02

(52) U.S. Cl. CPC A61K 31/423; A61K 31/4155; A61K 31/122; A61K 31/60; A61K 31/426; A61K 31/12; A61K 31/365; A61K 31/519; A61K 31/437; A61K 31/515; A61P 27/02

(57) ABSTRACT

Embodiments of the disclosure encompass methods and compositions for the treatment of medical conditions in which cell regeneration is in need, including neural cells. In specific embodiments, the cell are retinal cells and include Müller glial cells. In particular embodiments, an individual with retinal regeneration is provided an effective amount of one or more histone acetylase inhibitors for the purpose of regenerating retinal cells, including retinal neurons.

### CROSS-REFERENCE TO RELATED APPLICATIONS

This application claims priority to U.S. Provisional Patent Application Ser. No. 62/948254, filed Dec. 14, 2020, which is incorporated by reference herein in its entirety.

### STATEMENT REGARDING FEDERALLY SPONSORED RESEARCH OR DEVELOPMENT

This invention was made with government support under EY018571 awarded by the National Institutes of Health. The government has certain rights in the invention.

### TECHNICAL FIELD

Embodiments of the present disclosure concern at least the technical fields of cell biology, molecular biology, and medicine.

### BACKGROUND

Once damaged, either from injury or neural degenerative diseases, the central nervous system (CNS) has little spontaneous regeneration, repair or healing, resulting in permanent incapacity. Patients with serious nervous system defects often require lifelong assistance, putting a tremendous burden on the patient themselves, their families and society. Unfortunately, currently most of these conditions are without any effective cure. Therefore, innovative, paradigm-shifting strategies are required to advance treatment of neurological diseases. This disclosure provides new avenues of regenerative medicine by enabling reprogramming and differentiation of endogenous cells to replace damaged neurons in CNS.

As a model system, the disclosure encompasses use of the vision system, which is part of the CNS that is easily accessible. Vision plays a central role in the daily life of nearly every person, and blindness is one of the most feared disabilities in the population. Visual disorders affect more than 30 million people in the United States alone and the productivity cost of blindness worldwide is estimated to exceed 100 billion dollars per year. Degeneration of cells in the neural retina, the light sensing part of the visual system, because of injury or diseases lead to irreversible blindness. Although recent progress had been made in using gene therapy to treat inherited retinal diseases (IRD) at an early stage, no treatment is available for patients at mid- or late-stage of the disease whose retinal neural cells are already completely or largely degenerated. Currently, much of the research focus is to explore the potential of stem cell replacement therapy. Significant progress had been made in the past decade and it is now possible to obtain progenitor and differentiated retina neurons from either embryonic stem (ES) cells or induced pluripotent stem cells (iPSC). In addition, transplanted either progenitor cells or partially differentiated neuron to the retina in animal models show the potential to survive and integrate into the retina. However, several major challenges remain. First, the majority of donor cell die immediately after transplantation, which limits the efficacy and therapeutic potential. Second, based on results from animal models, very limited integration of the surviving donor cells into neuron circuitry has been achieved. Third, the ES cell derived cells are subject to immuno-rejection. As a result, long term immunosuppression is needed, putting the patient at risk of infection. Fourth, invasive surgery, such as subretinal injection, is needed to deliver the cells, further increasing the risk. Finally, there is reported risk of transplanted cells develop tumor or lead other adverse effects, such as retinal detachment, vitreous proliferation.

An alternate approach of using ES cells is to utilize endogenous cells in the retina, such as the Müller glia cells. It is well known that animals such as fish and birds can regenerate after retinal injury and replace the degenerated neurons and restore visual function. Unfortunately, this intrinsic regeneration capability is dramatically reduced in mammals, including mice and human, with only very few neurons being able to be generated upon damage. Therefore, although the regenerative machinery remains in the mammalian retina, the control of this intrinsic program responding to neuron degeneration is repressed. It is considered herein that if the regeneration program can be reactivated and controlled properly, it serves as the basis for developing new therapeutic approach in treating retinal degenerations, in at least some embodiments. In principle, intrinsic regeneration has several advantages over stem cell-based therapy. First, it is completely endogenous, therefore with no issue of immunologic rejection. Second, the outcome of intrinsic regeneration is significant where not only new neurons are born but also efficiently integrated into functional circuity, leading to fully restoration of function, a key challenging of the current stem cell-based method. Third, there is no need for performing invasive surgery. Currently, the approach to revive regeneration is mainly through ectopic expression of proneuronal genes. For example, it has been reported that over-expression of the proneuronal gene Ascll in Müller cells can promote the Müller cells to trans-differentiate into neuron-like cells, although the trans-differentiation is limited to amacrine and bipolar-like cells. It has recently been shown that by activating the Wnt signaling pathway followed by overexpression of a cocktail of transcription factors for rod photoreceptor cell development including Otx2, Crx and Nrl, a limited number of photoreceptor cells can be regenerated. Finally, it has been found that by knocking out the hippo pathway in Müller cells, Müller cells more readily enter the cell cycle, and a small fraction of them can differentiate into neuron-like cells upon injury. Unfortunately, the efficiency of regeneration and/or trans-differentiation is very limited (less than 1%) and most of the neurons born are not properly differentiated and integrated into circuity for all reports to date. In addition, neither the over-expression nor knock out method can lead to regeneration of all retinal cell types. Finally, because of the complex genetic manipulation needed, either through gene knock out and/or over expression, these approaches cannot be readily adapted into clinical therapy.

### BRIEF SUMMARY

The present disclosure is directed to methods and compositions for modulating mammalian genomes epigenetically to individuals in need thereof. The individual may have a need for regeneration of one or more cell types, including neural cells and in specific cases for retinal cells of any kind.

In particular embodiments, retinal regeneration is induced through methods encompassed herein by modulating epigenetic modifications via application (including in some cases transient application) of one or more small molecules and/or one or more proteins.

Embodiments of the disclosure encompass methods of treating an injury or neural degenerative disease in an individual, comprising the step of providing to the individual a therapeutically effective amount of one or more Histone Acetyltransferase inhibitors (HATi). In specific embodiments, methods of inducing regeneration of neuronal cells in a mammalian individual are provided, comprising the step of providing to the individual a therapeutically effective amount of one or more Histone Acetyltransferase inhibitors (HATi). In a certain embodiment, there are methods of treating hearing loss, spinal cord injury, brain injury due to trauma, shock, or a combination thereof, comprising the step of providing to the individual a therapeutically effective amount of one or more Histone Acetylation Transferase inhibitors (HATi). In specific embodiments, methods are provided of reducing the risk of retinal injury or disease, delaying the onset of retinal injury or disease, or reducing the severity of retinal injury or disease in an individual, comprising the step of providing to the individual a therapeutically effective amount of one or more Histone Acetylation Transferase inhibitors (HATi).

In any method of the disclosure, the Histone Acetyltransferase may be from the GNAT family, MYST family, p300/CBP family, or is Rtt109. Any HATi may or may not bind the active site of the histone acetyltransferase. In some cases, for any methods the HAT that is inhibited is Gcn5, PCAF, Hat1, Elp3, Hpa2, Hpa3, ATF-2, Nut1, MOZ, Ybf2 (Sas3), Sas2, Tip60, Esa1, MOF, MORF, HBO1, or a combination thereof. Any method of the disclosure may utilize a therapeutically effective amount of 2,3-bis(4-(furan-3-yl)phenyl)-5-(piperidin-4-ylmethoxy)pyrazine (which is referred to as SYC1127 elsewhere herein), A-485, C646, Garcinol, Anacardic acid, CPTH2, Curcumin, MB-3, Lys-CoA, H3-CoA-20, glycosaminoglycans, thiazinesulfonamide, isothiazolones, TH1634, ICG-001, benzylidenebarbituric acid, N1-aryl-propane-1,3-diamine derivative, cyclic peptide bromodomain derivatives, ischemin, N-phenyl (PU139) and N-benzyl (PU141) pyridoisothiazolones, tannic acid, BRD4, a functionally active derivative thereof, or a combination thereof.

In particular embodiments of any method of the disclosure, the injury or neurological disease affects the retina. In particular embodiments of any method of the disclosure, the neurological disease is the result of a genetic defect, such as a mutation in ADIPOR1, ARL6, BBIP1, BBS1, BBS2, BBS4, BBS5, BBS7, BBS9, BBS10, BBS12, C8orf37, CEP19, CEP290, IFT172, IFT27, INPP5E, KCNJ13, LZTFL1, MKKS, MKS1, NPHP1, SDCCAG8, TRIM32, TTC8, PRDM13, RGR, TEAD1, AIPL1, CRX, GUCA1A, GUCY2D, PITPNM3, PROM1, PRPH2, RIMS1, SEMA4A, UNC119, ABCA4, ADAMS, ATF6, C21orf2, C8orf37, CACNA2D4, CDHR1, CEP78, CERKL, CNGA3, CNGB3, CNNM4, GNAT2, IFT81, KCNV2, PDE6C, PDE6H, POC1B, RAB28, RAX2, RDH5, RPGRIP1, TTLL5, CACNA1F, RPGR, GNAT1, PDE6B, RHO, CABP4, GNAT1, GNB3, GPR179, GRK1, GRM6, LRIT3, RDH5, SAG, SLC24A1, TRPM1, CACNA1F, NYX, ESPN, WFS1, CDH23, CIB2, DFNB31, ESPN, MYO7A, PCDH15, PDZD7, USH1C, CRX, IMPDH1, OTX2, AIPL1, CABP4, CCT2, CEP290, CLUAP1, CRB1, CRX, DTHD1, GDF6, GUCY2D, IFT140, IQCB1, KCNJ13, LCA5, LRAT, NMNAT1, PRPH2, RD3, RDH12, RPE65, RPGRIP1, SPATA7, TULP1, BEST1, C1QTNF5, CTNNA1, EFEMP1, ELOVL4, FSCN2, GUCA1B, HMCN1, IMPG1, OTX2, PRDM13, PROM1, PRPH2, RP1L1, TIMP3, ABCA4, CFH, DRAM2, IMPG1, MFSD8, RPGR, VCAN, AFG3L2, MFN2, NR2F1, OPA1, ACO2, NBAS, RTN4IP1, TMEM126A, TIMM8A, ADIPOR1, ARL3, BEST1, CA4, CRX, FSCN2, GUCA1B, HK1, IMPDH1, IMPG1, KLHL7, NR2E3, NRL, PRPF3, PRPF4, PRPF6, PRPF8, PRPF31, PRPH2, RDH12, RHO, ROM1, RP1, RP9, RPE65, SAG, SEMA4A, SNRNP200, SPP2, TOPORS, ABCA4, AGBL5, AHR, ARHGEF18, ARL6, ARL2BP, BBS1, BBS2, BEST1, C2orf71, C8orf37, CERKL, CLCC1, CLRN1, CNGA1, CNGB1, CRB1, CYP4V2, DHDDS, DHX38, EMC1, EYS, FAM161A, GPR125, HGSNAT, IDH3B, IFT140, IFT172, IMPG2, KIAA1549, KIZ, LRAT, MAK, MERTK, MVK, NEK2, NEUROD1, NR2E3, NRL, PDE6A, PDE6B, PDE6G, POMGNT1, PRCD, PROM1, RBP3, REEP6, RGR, RHO, RLBP1, RP1, RP1L1, RPE65, SAG, SAMD11, SLC7A14, SPATA7, TRNT1, TTC8, TULP1, USH2A, ZNF408, ZNF513, OFD1, RP2, RPGR, ABCC6, AFG3L2, ATXN7, COL11A1, COL2A1, JAG1, KCNJ13, KIF11, MFN2, OPA3, PAX2, TREX1, VCAN, ABCC6, ABHD12, ACBD5, ACO2, ADAMTS18, ADIPOR1, AFG3L2, AHI1, ALMS1, CC2D2A, CEP164, CEP290, CLN3, COL9A1, CSPP1, ELOVL4, EXOSC2, FLVCR1, GNPTG, HARS, HGSNAT, HMX1, IFT140, IFT81, INPP5E, INVS, IQCB1, LAMA1, LRP5, MKS1, MTTP, NPHP1, NPHP3, NPHP4, OPA3, PANK2, PCYT1A, PEX1, PEX2, PEX7, PHYH, PLK4, PNPLA6, P005, POC1B, PRPS1, RDH11, RPGRIP1L, SDCCAG8, SLC25A46, TMEM216, TMEM237, TRNT1, TTPA, TUB, TUBGCP4, TUBGCP6, WDPCP, WDR19, WFS1, ZNF423, OFD1, TIMM8A, ABHD12, ADGRV1, ARSG, CDH23, CEP250, CEP78, CIB2, CLRN1, DFNB31, ESPN, HARS, MYO7A, PCDH15, USH1C, USH1G, USH2A, BEST1, CAPN5, CRB1, ELOVL1, FZD4, ITM2B, LRP5, MAPKAPK3, MIR204, OPN1SW, RB1, RCBTB1, TSPAN12, ZNF408, ASRGL1, BEST1, C12orf65, CDH3, CNGA3, CNGB3, CNNM4, CYP4V2, LRP5, MFRP, MVK, NBAS, NR2E3, OAT, PLA2G5, PROM1, RBP4, RCBTB1, RGS9, RGS9BP, RLBP1, KSS, LHON, MT-ATP6, MT-TH, MT-TL1, MT-TP, MT-TS2, CACNA1F, CHM, DMD, NDP, OPN1LW, OPN1MW, PGK1, RS1, ABCA4, ARMS2, C2, C3, CFB, CFH, ERCC6, FBLN5, HMCN1, HTRA1, RAX2, TLR3, TLR4, or a combination thereof.

In any methods of the disclosure concerning the retina, the method may result in regeneration of cells in the retina selected from the group consisting of retinal ganglion cells, photoreceptor cells, amacrine, bipolar cells, Müller cells, horizontal cells, and a combination thereof. Any injury may be from hazardous radiation, physical force or intrusion, a retinal tear, retinal detachment, damaged induced by chemical agent(s), damage induced by neurotoxic agent(s), or a combination thereof. An individual may have retinal injury and/or degenerative retinal neurons.

In specific embodiments, the methods treat any neural degenerative disease, including a disease of the central nervous system, such as one selected from the group consisting of Alzheimer's disease, amyotrophic lateral sclerosis, Friedreich's ataxia, Huntington's disease, Lewy body disease, Parkinson's disease, Spinal muscular atrophy, and a combination thereof.

In some methods of the disclosure, the individual is at risk for any neural degenerative disease. Any individual treated by any method encompassed herein may have diabetes, cardiovascular disease, high blood pressure, high blood sugar, being a smoker, excessive ultraviolet light exposure; high myopia, a personal history, and/or a family history.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1C concern an example of retinal regeneration in Spata7-mutated (KO) retinal cells. (1A) One example of a timeline for studies, where the examples of timepoints are post-natal day 14, 21, and 34, respectively. (1B) Hematoxylin and eosin (H&E) staining for Spata7 KO mice versus wild-type control. Eyes were injected with SYC1127 (treated) eye compared to PBS-injected control. (1C) Immunofluorescence staining for Cone-rod homeobox protein (CRX, a marker for photoreceptor cells and bipolar cells); Glial fibrillary acidic protein (GFAP, a marker for Müller glial cells); and RNA Binding Protein, MRNA Processing Factor (RBPMS, a marker for retinal ganglion cells (RGC)) in Spata7 KO-treated eye (treated with SYC1127) and control eye (treated with PBS).

FIG. 2 shows that intravitreal injection of SYC1127 alters chromatin modification in the retina. The top row demonstrates H&E staining for control (PBS buffer-injected) or injection of SYC1127 in wild-type retina. The outer nuclear layers (ONL) and the inner nuclear layers (INL) of the respective retina are shown. Markers related to reduced levels of histone acetylation (H3K27Ac) and increased levels of histone acetylation (H3K9Me3) are identified with corresponding antibodies.

FIG. 3 shows retinal regeneration induced through epigenomics manipulation. H&E staining of all three neural layers of the retina is shown following intravitreal injection of SYC1127 in wild-type mice vs. Spata7 KO mutants.

FIG. 4 demonstrates induction of retinal ganglion cells following application of SYC1127 (compared to PBS control), including with H&E staining (top row) and immunohistochemistry with fluorescent-tagged antibodies that bind RBPMS (bottom row).

FIG. 5 demonstrates evidence that retinal regeneration is driven by Müller Glial Cells. Immunohistochemistry using antibodies that bind Sox9 or GFAP, both markers of Müller glial cells, is provided for SYC1127-treated Spata7 KO mutants vs. PBS controls.

### DETAILED DESCRIPTION

In keeping with long-standing patent law convention, the words “a” and “an” when used in the present specification in concert with the word comprising, including the claims, denote “one or more.” As used in the specification and claims, the singular form “a”, “an”, and “the” include plural references unless the context clearly dictates otherwise. For example, the term “a nucleic acid” includes a plurality of nucleic acids, including mixtures thereof. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “subject,” as used herein, generally refers to an individual in need of treatment, including for a medical condition in which cell regeneration is therapeutic. The subject can be any animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more neurological diseases, one or more diseases or disorders associated with the retina, or any combination thereof. The disease may or may not be pathogenic, acute, or chronic. The subject may be asymptomatic. The term “individual” may be used interchangeably, in at least some embodiments. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants and includes in utero individuals. A subject may or may not have a need for medical treatment; an individual may voluntarily or involuntarily be part of experimentation whether clinical or in support of basic science studies.

The term “therapeutically effective amount” refers to the amount of a HATi with or without additional agents that is effective to achieve its intended purpose. While individual patient needs may vary, determination of optimal ranges for effective amounts of each of the compounds and compositions is within the skill of an ordinary practitioner of the art. Generally, the dosage required to provide an effective amount of the composition, and which can be adjusted by one of ordinary skill in the art, will vary, depending on the age, health, physical condition, sex, weight, extent of the dysfunction of the recipient, frequency of treatment and the nature and scope of the dysfunction.

Once damaged, either from injury or neural degenerative diseases, the central nervous system including the retina has little spontaneous regeneration, repair, or healing, resulting in permanent incapacity. With respect to the retina, the present disclosure concerns enablement of reprogramming and differentiation of, e.g., endogenous Müller glia cells to replace degenerated neurons in the retina. The disclosure also provides methods and compositions for highly efficient neural regeneration.

I. Compositions

Embodiments of the disclosure include compositions useful for regeneration of cells, including cells of neural tissue. In particular embodiments, the compositions are utilized for regeneration of cells in the retina, and such compositions in specific embodiments includes one or more Histone Acetyltransferase inhibitors (HATi).

Histone acetyltransferases (HAT) are enzymes that acetylate conserved lysine amino acids on histone proteins. They do so by transferring an acetyl group from acetyl CoA to form e-N-acetyl lysine. Any inhibitors of HAT may be used that may directly or indirectly inhibit the activity and/or expression of any one or more HAT.

Any HATi may be utilized in any embodiments of the disclosure. One or more HATi may be utilized in one or more methods of the disclosure. Although the HATi may be of any kind, in particular embodiments the HATi is of a particular family, such as of the Gcn5-related N-acetyltransferases (GNAT) family, MYST family, p300/CBP family, or is Rtt109. Examples of GNAT HATs that may be targeted by the inhibitors includes Gcn5, PCAF, Hat1, Elp3, Hpa2, Hpa3, ATF-2, and Nut1. The HATi may target any one of Gcn5, PCAF, Hat1, Elp3, Hpa2, Hpa3, ATF-2, and Nut1. Examples of MYST family members includes MOZ, Ybf2 (Sas3), Sas2, Tip60 (the family is named after these first four members), Esa1, MOF, MORF, and HBO1.

In certain embodiments, the HATi comprise bi-substrate inhibitors, natural product derivatives, small molecules, and/or protein-protein interaction inhibitors

In cases wherein the HATi directly bind the HAT, the HATi may or may not bind the active site of the histone acetylation transferase. The HAT that is inhibited may be a Type A HAT or a Type B HAT.

In specific embodiments, the HATi is 2,3-bis(4-(furan-3-yl)phenyl)-5-(piperidin-4-ylmethoxy)pyrazine (SYC1127), A-485, C646, Garcinol, Anacardic acid, CPTH2, Curcumin, MB-3, Lys-CoA, H3-CoA-20, glycosaminoglycans, thiazinesulfonamide, isothiazolones, TH1634, ICG-001, benzylidenebarbituric acid, N1-aryl-propane-1,3-diamine derivative, cyclic peptide bromodomain derivatives, ischemin, N-phenyl (PU139) and N-benzyl (PU141) pyridoisothiazolones, tannic acid, BRD4, functionally active derivatives thereof (having histone acetyltransferase activity), or a combination thereof. Histone acetyltransferase assays are commercially available; examples of assays include Fluorometric (thiol-scavenging probe); Enzyme-coupling; Radiolabeled substrate (filter-binding); Alpha technology (AlphaScreen, AlphaLISA); FRET, and so forth.

One or more HATi compounds can be provided in a composition for use in various methods of the disclosure, which are described herein. Pharmaceutically acceptable salts of the HATi(s) may be used so long as they do not adversely affect its activity. Particular salts may be selected and made by those skilled in the art. For example, an alkali metal salt, such as a sodium salt or a potassium salt, an alkaline earth metal salt, such as a calcium salt or a magnesium salt, may be used. Similarly, a salt with an inorganic base, such as an ammonium salt, or a salt with an organic base, such as a triethylamine salt or an ethanolamine salt, may be used.

The HATi compositions can comprise a pharmaceutically acceptable carrier, including, but not limited to, saline, buffered saline or other physiologically compatible buffers, dextrose, and water. In specific embodiments, the pharmaceutically acceptable carrier is non-pyrogenic, most preferably it is sterile. If embodiments wherein the HATi is a protein or nucleic acid, the HATi compositions can comprise an expression construct encoding the protein or nucleic acid. In the expression construct, transcription of a coding sequence for the desired polypeptide is under the control of a promoter, such as an SV40, CMV, ADH1, T7, or T3 promoter. If the expression construct encodes more than one HATi gene product (protein or nucleic acid), each can be under the control of a separate promoter or one promoter can control transcription of two or more coding sequences. Many expression constructs suitable for inclusion in HATi-containing compositions are commercially available or can be easily constructed using well-known methods, such as recombinant DNA techniques and synthetic techniques.

Upon delivery of the one or more HATi to the desired location in an individual, cells or tissue in need of regeneration are provided an effective amount of the HATi(s) to be able to induce regeneration. In particular embodiments, the activity of the HATi(s) is to inhibit activity of the histone acetylase relative to activity in the absence of the HATi(s). Histone acetylase activity may be inhibited at least 10, 25, 30, 40, 50, 60, 70, 75, 80, 90, 95, 97, 99, or 100% relative to activity in the absence of the HATi(s).

In cases wherein more than one HATi is to be provided to an individual in need thereof, the multiple HATi may or may not be present in the same composition. At the time of delivery, the multiple HATi may be present in the formulation and may or may not be stored prior to delivery in the same container. In cases wherein the multiple HATi are to be delivered to an individual, and they are in separate delivery vessels, they may be delivered in any order. A first and second HATi may be delivered separately for a particular delivery to the individual and together for a different delivery to the individual.

II. Methods of Use

Methods of the disclosure include methods for the regeneration of cells, including cells of neural tissue, such as cells of neural tissue in the retina, including neurons. In particular embodiments, the compositions are utilized for regeneration of cells of any kind in the retina. Methods of the disclosure may encompass particular biological active steps for the purpose of regeneration of cells, such as through chromatin modifications and/or epigenomics manipulation. In specific embodiments, regeneration of cells in the retina is directly or indirectly induced by Müller glial cells present in the retina. In specific cases, proliferation of Müller glial cells precedes the regeneration of other cells in the retina, such as neurons in the retina.

Specific embodiments of the disclosure include inducing intrinsic regeneration of cells of any kind, including at least Müller cells.

In particular embodiments, the methods of the disclosure include methods of regenerating cells in any layer of neurons in the neural retina, including the outer layer of photoreceptors, a middle layer of bipolar/amacrine/horizontal neurons, and/or an inner layer of ganglion cells. In certain embodiments, the methods encompass regeneration of at least the outer layer and middle layer, at least the outer layer and inner layer, or at least the middle layer and inner layer.

In certain embodiments any particular type of retinal cells may be regenerated through methods of the disclosure including at least Müller cells, photoreceptor cells, bipolar cells, retinal ganglion cells, amacrine cells, horizontal cells, or a combination thereof.

In some embodiments, the presence, the location and/or extent of a retinal disease or injury is assayed by one or more tests, such as Amsler grid test; Optical coherence tomography (OCT); Fundus autofluorescence (FAF); Fluorescein angiography; Indocyanine green angiography; Ultrasound.; CT; and/or MRI.

In some embodiments, an individual seeking treatment for a retinal disease or injury is provided an effective amount of a therapy other than the HATi therapy of the disclosure, such as laser surgery; scatter laser photocoagulation; cryopexy; pneumatic retinopexy; scleral buckling; vitrectomy; injection of a non-HATi drug, such as into the vitreous in the eye; and/or retinal prosthesis. In cases wherein the individual in need of HATi therapy is provided one or more other retinal therapies, the second or more other therapy may be provided before, during, and/or after one or more HATi. An individual may receive both laser surgery and one or more HATi at the same time or at different times. For example, an individual may receive laser surgery for an immediate (sometimes urgent) treatment, followed by one or more HATi, including for long-term repair and maintenance of the retina.

Examples of retinal diseases in which retinal regeneration may be therapeutic include at least retina tear; retinal detachment; retinal degeneration with age; diabetic retinopathy; epiretinal membrane; macular hole; macular degeneration; retinitis pigmentosa; or a combination thereof. In some embodiments, an individual is provide an effective amount of one or more HATi because they are at risk for a retinal disease or injury, such as at risk because of a prior retinal disease or injury, a family history of retinal disease, and so forth. In some cases, an individual is provided an effective amount of one or more HATi because they are at risk for retinal disease from having diabetes, cardiovascular disease, high blood pressure, high blood sugar, being a smoker, excessive ultraviolet light exposure; and/or having high myopia. In such cases, an individual may be provided an effective amount of one or more HATi prior to the onset of the retinal disease or injury. Such individuals may have prevention of retinal disease or injury, delay in onset, delay in severity, or delay in both onset and delivery, for example.

In some embodiments, the regeneration phenotype may be systematically characterized. One can characterize the time course of cell proliferation and/or differentiation. For example, upon SYC1127 injection, daily injection of BrdU may be performed to characterize cell proliferation curve. Retinal cell type-specific markers may be co-stained with BrdU and Müller glial cell markers to identify the regeneration of retinal cell types upon SYC1127 injection. In some embodiments, one can identify the source of any of the regenerative cells, and in specific embodiments Müller glial cells are the source of regeneration. In a specific example, one can induce regeneration in GLAST-creER (Jackson stock 012586); Ai9 mice where Müller cells are positively labelled with tdTomato. Newly generated cells would be tdTomato positive if they are derived from Müller Cells.

In certain embodiments, the function of the regenerated cells is characterized. Preliminary data indicates that the regenerated cells integrate properly into the correct layers. One can examine if newly generated cells have proper morphology and/or form proper synapse with other cells. One can examine if newly generated cells function normally by performing electrophysiology testing, for example.

In embodiments, one can investigate the potential of epigenetic modulation in regenerative therapy. In specific cases, one can examine if regeneration can be induced broadly under different degeneration conditions. An example is provided in the table immediately below:

| Condition | Type | Target Cells |
|---|---|---|
| Spata7 KO | Genetics | Photoreceptor Cells |
| N-methyl-D-aspartate | excitatory | RGCs and inner |
| (NMDA) | neurotoxicity | nuclear layer neurons |
| N-methyl-Nnitrosourea | Chemical | Photoreceptor cells |
| (NMN) | | |
| Optic nerve crush | Mechanical | RGCs |
| | injury | |

In embodiments, one can implement a mechanistic study, including why and/or how the regeneration is induced. In specific embodiments, one can confirm the causal relationship between epigenetic modulation and regeneration. In specific embodiments, one can identify key changes in the chromatin state and gene expression. In a certain example, single cell RNAseq and single cell ATACseq, e.g., can be performed to profile the regenerative retina. Common changes across different degeneration model and drug treatment may be identified for follow up studies.

In particular embodiments, epigenetic modulation is utilized as a novel regenerative therapeutic method. In specific embodiments, any one of more HATi are utilized for regeneration of cells, including neural cells, and including retinal cells. In specific cases, SYC1127 is utilized as a treatment for an individual in need, including an individual in need of cell regeneration, such as neural cell regeneration and including retinal cell regeneration.

In certain embodiments, one can identify and further optimize small molecules that target the same or different epigenetic modification enzymes, including HAT. Based on the present disclosure, one can test if similar approaches can be applied to other CNS organs, for example.

Non-Retinal Embodiments

In certain embodiment, the disclosure encompasses methods and compositions for treating diseases other than for the retina. The treatment may concern an injury or disease of the CNS, including, for example any neural degenerative disease. The neural degenerative disease may or may not be the result of a genetic defect in an individual such as having one or more mutations in ADIPOR1, ARL6, BBIP1, BBS1, BBS2, BBS4, BBS5, BBS7, BBS9, BBS10, BBS12, C8orf37, CEP19, CEP290, IFT172, IFT27, INPP5E, KCNJ13, LZTFL1, MKKS, MKS1, NPHP1, SDCCAG8, TRIM32, TTC8, PRDM13, RGR, TEAD1, AIPL1, CRX, GUCA1A, GUCY2D, PITPNM3, PROM1, PRPH2, RIMS1, SEMA4A, UNC119, ABCA4, ADAMS, ATF6, C21orf2, C8orf37, CACNA2D4, CDHR1, CEP78, CERKL, CNGA3, CNGB3, CNNM4, GNAT2, IFT81, KCNV2, PDE6C, PDE6H, POC1B, RAB28, RAX2, RDH5, RPGRIP1, TTLL5, CACNA1F, RPGR, GNAT1, PDE6B, RHO, CABP4, GNAT1, GNB3, GPR179, GRK1, GRM6, LRIT3, RDH5, SAG, SLC24A1, TRPM1, CACNA1F, NYX, ESPN, WFS1, CDH23, CIB2, DFNB31, ESPN, MYO7A, PCDH15, PDZD7, USH1C, CRX, IMPDH1, OTX2, AIPL1, CABP4, CCT2, CEP290, CLUAP1, CRB1, CRX, DTHD1, GDF6, GUCY2D, IFT140, IQCB1, KCNJ13, LCA5, LRAT, NMNAT1, PRPH2, RD3, RDH12, RPE65, RPGRIP1, SPATA7, TULP1, BEST1, C1QTNF5, CTNNA1, EFEMP1, ELOVL4, FSCN2, GUCA1B, HMCN1, IMPG1, OTX2, PRDM13, PROM1, PRPH2, RP1L1, TIMP3, ABCA4, CFH, DRAM2, IMPG1, MFSD8, RPGR, VCAN, AFG3L2, MFN2, NR2F1, OPA1, ACO2, NBAS, RTN4IP1, TMEM126A, TIMM8A, ADIPOR1, ARL3, BEST1, CA4, CRX, FSCN2, GUCA1B, HK1, IMPDH1, IMPG1, KLHL7, NR2E3, NRL, PRPF3, PRPF4, PRPF6, PRPF8, PRPF31, PRPH2, RDH12, RHO, ROM1, RP1, RP9, RPE65, SAG, SEMA4A, SNRNP200, SPP2, TOPORS, ABCA4, AGBL5, AHR, ARHGEF18, ARL6, ARL2BP, BBS1, BBS2, BEST1, C2orf71, C8orf37, CERKL, CLCC1, CLRN1, CNGA1, CNGB1, CRB1, CYP4V2, DHDDS, DHX38, EMC1, EYS, FAM161A, GPR125, HGSNAT, IDH3B, IFT140, IFT172, IMPG2, KIAA1549, KIZ, LRAT, MAK, MERTK, MVK, NEK2, NEUROD1, NR2E3, NRL, PDE6A, PDE6B, PDE6G, POMGNT1, PRCD, PROM1, RBP3, REEP6, RGR, RHO, RLBP1, RP1, RP1L1, RPE65, SAG, SAMD11, SLC7A14, SPATA7, TRNT1, TTC8, TULP1, USH2A, ZNF408, ZNF513, OFD1, RP2, RPGR, ABCC6, AFG3L2, ATXN7, COL11A1, COL2A1, JAG1, KCNJ13, KIF11, MFN2, OPA3, PAX2, TREX1, VCAN, ABCC6, ABHD12, ACBD5, ACO2, ADAMTS18, ADIPOR1, AFG3L2, AHI1, ALMS1, CC2D2A, CEP164, CEP290, CLN3, COL9A1, CSPP1, ELOVL4, EXOSC2, FLVCR1, GNPTG, HARS, HGSNAT, HMX1, IFT140, IFT81, INPP5E, INVS, IQCB1, LAMA1, LRP5, MKS1, MTTP, NPHP1, NPHP3, NPHP4, OPA3, PANK2, PCYT1A, PEX1, PEX2, PEX7, PHYH, PLK4, PNPLA6, P005, POC1B, PRPS1, RDH11, RPGRIP1L, SDCCAG8, SLC25A46, TMEM216, TMEM237, TRNT1, TTPA, TUB, TUBGCP4, TUBGCP6, WDPCP, WDR19, WFS1, ZNF423, OFD1, TIMM8A, ABHD12, ADGRV1, ARSG, CDH23, CEP250, CEP78, CIB2, CLRN1, DFNB31, ESPN, HARS, MYO7A, PCDH15, USH1C, USH1G, USH2A, BEST1, CAPN5, CRB1, ELOVL1, FZD4, ITM2B, LRP5, MAPKAPK3, MIR204, OPN1SW, RB1, RCBTB1, TSPAN12, ZNF408, ASRGL1, BEST1, C12orf65, CDH3, CNGA3, CNGB3, CNNM4, CYP4V2, LRP5, MFRP, MVK, NBAS, NR2E3, OAT, PLA2G5, PROM1, RBP4, RCBTB1, RGS9, RGS9BP, RLBP1, KSS, LHON, MT-ATP6, MT-TH, MT-TL1, MT-TP, MT-TS2, CACNA1F, CHM, DMD, NDP, OPN1LW, OPN1MW, PGK1, RS1, ABCA4, ARMS2, C2, C3, CFB, CFH, ERCC6, FBLN5, HMCN1, HTRA1, RAX2, TLR3, or TLR4.

In cases wherein the non-retinal disease is of the central nervous system, such as a neural degenerative disease, the disease may be selected from the group consisting of Alzheimer's disease, amyotrophic lateral sclerosis, Friedreich's ataxia, Huntington's disease, Lewy body disease, Parkinson's disease, Spinal muscular atrophy, and a combination thereof. The individual may be at risk for the neural degenerative disease because of a personal or family history. In such cases, the individual may be provided an effective amount of one or more HATi whether or not a symptom of the neural degenerative disease has manifested.

In some embodiments, there are methods of treating hearing loss, spinal cord injury, brain injury due to trauma, shock, or a combination thereof, by administering to the individual an effective amount of one or more HATi. The HATi(s) may be provided to an individual at risk for hearing loss, spinal cord injury, brain injury of any kind, and/or shock, such as to delay the onset, delay the severity, or delay both onset and delivery.

III. Pharmaceutical Preparations

Pharmaceutical compositions of the present disclosure comprise an effective amount of one or more HATi dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one HATi will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated. The carrier may be particularly suited for intravitreal delivery, such as a syringe for injection. Intravitreal delivery, or any delivery, may include one or more antibiotics whether or not in the same formulation or at the same time.

The HATi(s) may comprise different types of carriers depending on whether it is to be administered in solid, liquid or aerosol form, and whether it need to be sterile for such routes of administration as injection. The present disclosure can be administered intravitreally, although in alternative cases it is administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The HATi(s) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include HATi(s), one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the HATi(s) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In specific aspects for dosage, a concentration may be from 1-200 μM or any range derivable therein. A concentration may be in the range of 1-200, 1-175, 1-150, 1-125, 1-100, 1-75, 1-50, 1-25, 1-10, 10-200, 10-175, 10-150, 10-125, 10-100, 10-75, 10-50, 10-25, 25-200, 25-175, 25-150, 25-125, 25-100, 25-75, 25-50, 50-200, 50-175, 50-150, 50-125, 50-100, 50-75, 75-200, 75-175, 75-150, 75-125, 75-100, 100-200, 100-175, 100-150, 100-125, 125-200, 125-175, 125-150, 150-200, 150-175, or 175-200 μM. The concentration may be, or may be about, 1, 10, 25, 50, 75, 100, 125, 150, 175, or 200 μM.

A volume for delivery may be from 25-100 μl including 25-100, 25-75, 25-50, 50-100, 50-75, or 75-100 μl for example. The volume for delivery may be 25, 50, 75, or 100 μl.

In particular embodiments, delivery is through local injection at or around the site needing repair. In some cases, delivery is through other methods, such as through blood circulation. In certain aspects, the HATi is able to pass the blood-brain barrier.

A. Alimentary Compositions and Formulations

In particular embodiments of the present disclosure, the HATi(s) are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft- shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, one or more HATi(s) may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be useful to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other particular embodiments of the disclosure, the active compound HATi(s) may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

IV. Kits of the Disclosure

Any of the HATi compositions described herein may be comprised in a kit in suitable container means.

The kits may comprise one or more suitably aliquoted HATi and optionally one or more additional agents. The components of the kits may be suitably packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the HATi and any other reagent containers in close confinement for commercial sale. Such containers may include injection or blow-molded plastic containers into which the desired vials are retained.

In cases wherein more than one HATi are included in the kit, the multiple HATi may or may not be in the same container. When the component(s) of the kit are provided in one and/or more liquid solutions, the liquid solution is an aqueous solution, with a sterile aqueous solution being particularly preferred. The HATi compositions may also be formulated into a syringeable composition. In which case, the container means may itself be a syringe, pipette, and/or other such like apparatus, from which the formulation may be applied to an infected area of the body (including an eye), injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means, although in some cases the user may provide such solvent.

In certain embodiments, the kit comprises one or more additional agents for treatment of the medical condition for which the HATi(s) are being utilized. In some embodiments, the kit comprises one or more apparatuses and/or one or more reagents for diagnosis of a medical condition for which the HATi(s) are being utilized.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Chromatin Modifications in Retinal Cells and Regeneration Thereof

In the present disclosure, a novel approach is provided to reactivate the intrinsic regeneration program. Dramatic epigenetic changes occur during retinal degeneration, causing cells to become partially de-differentiated and transcriptionally repressed. Based on this observation, it was considered that further inducing Muller cells into a repressive state through regulating epigenetics modifies enzyme activity, and this would lead to activation of an intrinsic program.

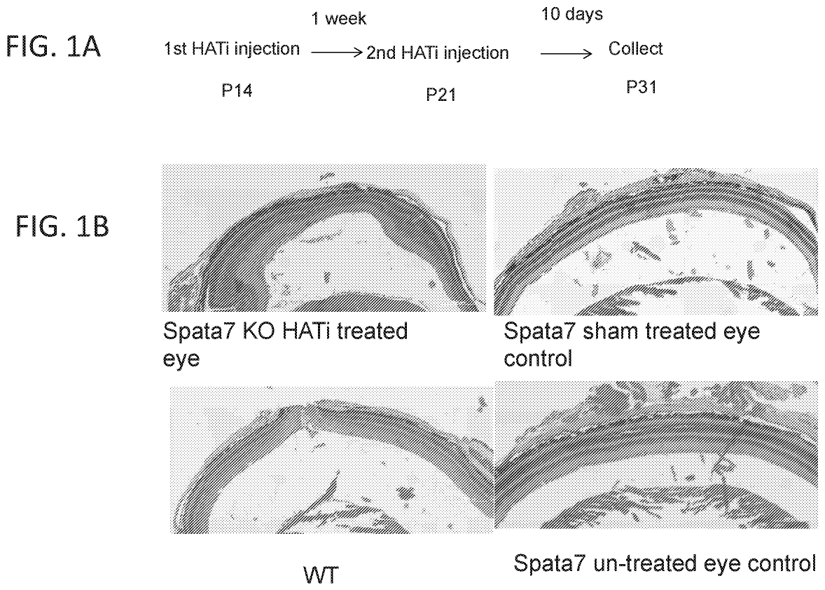

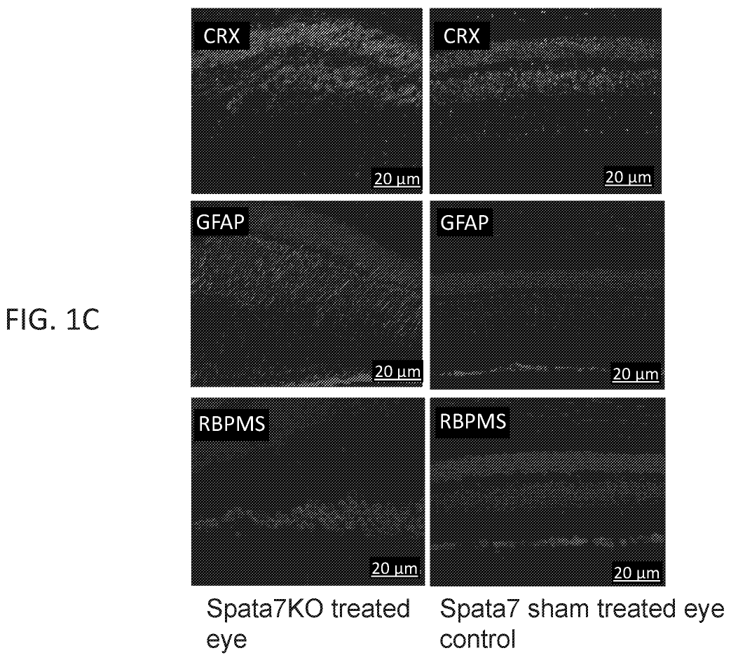

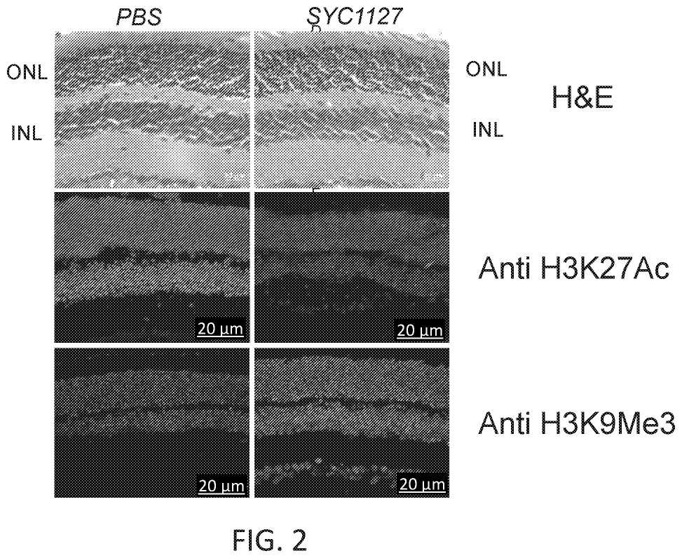

In this present example, one example of a potent Histone Acetylation Transferase inhibitor (HATi), the compound SYC1127, is administered by intravitreal injection in a retinal degeneration mouse model having a knockout mutation in Spata7. An example of an injection regimen is provided in FIG. 1A, in which there are multiple intravitreal injections. As shown in FIG. 1B, injection of the drug is not toxic, and little change is observed in the wild-type control. Strikingly, extensive cell proliferation is observed when Spata7 mutant retina are injected with SYC1127. Compared to the uninjected Spata7 mutant retina, thickening of all three neuronal layers (the neural retina comprises three layers of neurons—the outer layer of photoreceptors, a middle layer of bipolar neurons, and an inner layer of ganglion cells that collect visual signals and carry them to the optic nerve via elongated cell processes) are observed, indicating the generation of all retinal neuronal cell types. To examine if these newly generated cells are indeed differentiated properly, immunohistochemistry (IHC) using antibodies against certain cell type-specific markers is performed. As shown in FIG. 1C, multiple layers of cells that are positive for retinal ganglion cells (RGC) are observed, clearly indicating the generation of new RGCs in treated retina. Similar results have been obtained from both the photoreceptor cells (FIG. 1C), amacrine, and bipolar cells, indicating a total regeneration. These newly generated neurons are likely derived from Müller glia cells, as a massive elevated Müller glia cell activation is observed (as shown highlighted by GFAP staining). In conclusion, by pushing the cell into a repressive mode via a HATi, a complete regeneration of retinal neurons in degenerating retina can be achieved. The number of regenerative cells is much higher than that of previously reported by others, making it useful for therapy.

Further evidence is provided that HATi are useful for chromatin modifications that lead to epigenomic manipulation, thereby leading to regeneration of retinal cells. FIG. 2 shows that intravitreal injection of SYC1127 alters chromatin modification in the retina. Upon intravitreal injection of the compound SYC1127, there is reduction in the level of histone acetylation as expected, such as with H3K27Ac (an epigenetic modification to Histone H3 that is a mark that indicates the acetylation at the 27th lysine residue of the Histone H3 protein). At the same time, the level of chromatin-repressing histone markers, such as H3K9Me3, is increased.

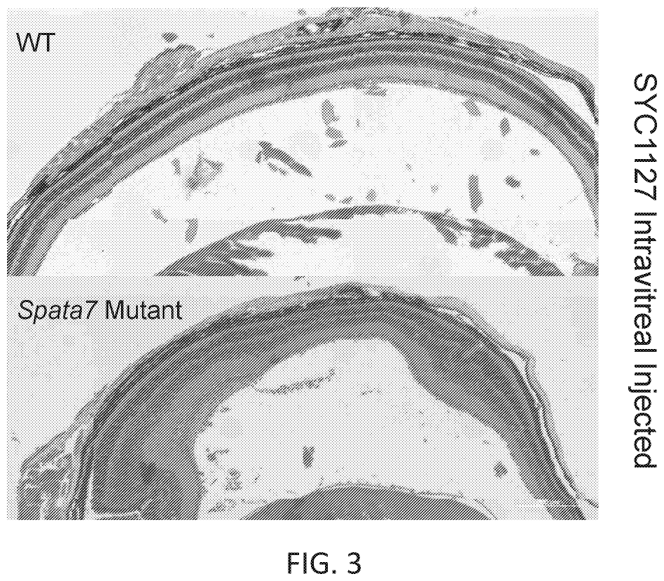

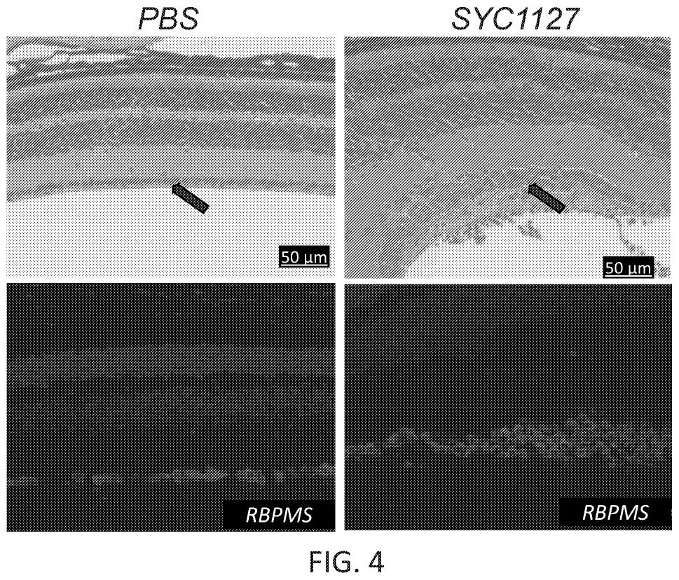

Retinal regeneration is induced through epigenomics manipulation, in particular embodiments. As shown in FIG. 3, intravitreal injection of SYC1127 into Spata7 mutants leads to proliferation of all three neural layers of the retina. In contrast, no changes are observed when SYC1127 is applied to the wild-type control retina. As at least one type of retinal cells, retinal ganglion cells are induced following administration of SYC1127. FIG. 4 demonstrates induction of retinal ganglion cells, in which case the increase of cells in the RGC layer (red arrow) upon applying HATi is observed, as indicated with positive RBPMS staining.

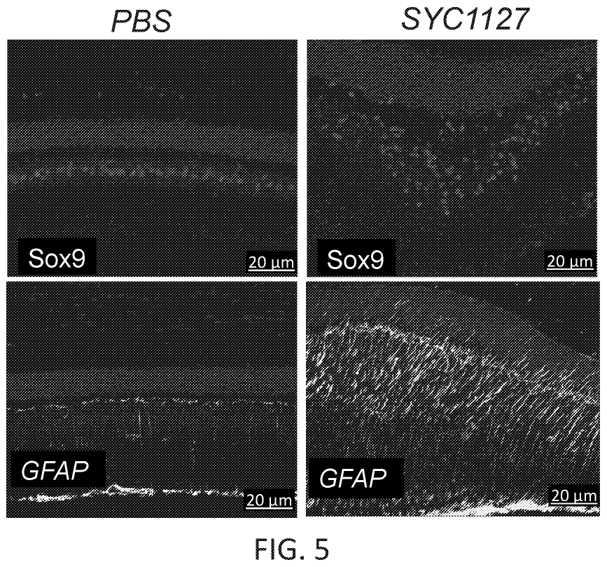

FIG. 5 demonstrates evidence that retinal regeneration is driven by Müller glial cells. Increase in the number of Müller glial cells is observed upon treatment as shown by increasing of Sox9 positive cells and GFAP staining. Migration of Müller glial cells from the INL to photoreceptor and retinal ganglion cell layers is observed.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of treating an injury or neural degenerative disease in an individual, comprising the step of providing to the individual a therapeutically effective amount of one or more Histone Acetyltransferase inhibitors (HATi).

2. The method of claim 1, wherein the Histone Acetyltransferase is from the GNAT family, MYST family, p300/CBP family, or is Rtt109.

3. The method of claim 1, wherein the HATi binds the active site of the histone acetyltransferase.

4. The method of claim 1, wherein the HATi does not bind the active site of the histone acetyltransferase.

5. The method of claim 1, wherein the HAT that is inhibited is Gcn5, PCAF, Hat1, Elp3, Hpa2, Hpa3, ATF-2, Nut1, MOZ, Ybf2 (Sas3), Sas2, Tip60, Esa1, MOF, MORF, HBO1, or a combination thereof.

6. The method of claim 1, wherein the HATi is SYC1127, A-485, C646, Garcinol, Anacardic acid, CPTH2, Curcumin, MB-3, Lys-CoA, H3-CoA-20, glycosaminoglycans, thiazinesulfonamide, isothiazolones, TH1634, ICG-001, benzylidenebarbituric acid, N1-aryl-propane-1,3-diamine derivative, cyclic peptide bromodomain derivatives, ischemin, N-phenyl (PU139) and N-benzyl (PU141) pyridoisothiazolones, tannic acid, BRD4, a functionally active derivative thereof, or a combination thereof.

7. The method of claim 1, wherein the injury or neurological disease affects the retina.

8. The method of claim 1, wherein the neurological disease is the result of a genetic defect.

9. The method of claim 8, wherein the genetic defect is a mutation in ADIPOR1, ARL6, BBIP1, BBS1, BBS2, BBS4, BBS5, BBS7, BBS9, BBS10, BBS12, C8orf37, CEP19, CEP290, IFT172, IFT27, INPP5E, KCNJ13, LZTFL1, MKKS, MKS1, NPHP1, SDCCAG8, TRIM32, TTC8, PRDM13, RGR, TEAD1, AIPL1, CRX, GUCA1A, GUCY2D, PITPNM3, PROM1, PRPH2, RIMS1, SEMA4A, UNC119, ABCA4, ADAM9, ATF6, C2lorf2, C8orf37, CACNA2D4, CDHR1, CEP78, CERKL, CNGA3, CNGB3, CNNM4, GNAT2, IFT81, KCNV2, PDE6C, PDE6H, POC1B, RAB28, RAX2, RDH5, RPGRIP1, TTLL5, CACNA1F, RPGR, GNAT1, PDE6B, RHO, CABP4, GNAT1, GNB3, GPR179, GRK1, GRM6, LRIT3, RDH5, SAG, SLC24A1, TRPM1, CACNA1F, NYX, ESPN, WFS1, CDH23, CIB2, DFNB31, ESPN, MYO7A, PCDH15, PDZD7, USH1C, CRX, IMPDH1, OTX2, AIPL1, CABP4, CCT2, CEP290, CLUAP1, CRB1, CRX, DTHD1, GDF6, GUCY2D, IFT140, IQCB1, KCNJ13, LCA5, LRAT, NMNAT1, PRPH2, RD3, RDH12, RPE65, RPGRIP1, SPATA7, TULP1, BEST1, C1QTNF5, CTNNA1, EFEMP1, ELOVL4, FSCN2, GUCA1B, HMCN1, IMPG1, OTX2, PRDM13, PROM1, PRPH2, RP1L1, TIMP3, ABCA4, CFH, DRAM2, IMPG1, MFSD8, RPGR, VCAN, AFG3L2, MFN2, NR2F1, OPA1, ACO2, NBAS, RTN4IP1, TMEM126A, TIMM8A, ADIPOR1, ARL3, BEST1, CA4, CRX, FSCN2, GUCA1B, HK1, IMPDH1, IMPG1, KLHL7, NR2E3, NRL, PRPF3, PRPF4, PRPF6, PRPF8, PRPF31, PRPH2, RDH12, RHO, ROM1, RP1, RP9, RPE65, SAG, SEMA4A, SNRNP200, SPP2, TOPORS, ABCA4, AGBL5, AHR, ARHGEF18, ARL6, ARL2BP, BBS1, BBS2, BEST1, C2orf71, C8orf37, CERKL, CLCC1, CLRN1, CNGA1, CNGB1, CRB1, CYP4V2, DHDDS, DHX38, EMC1, EYS, FAM161A, GPR125, HGSNAT, IDH3B, IFT140, IFT172, IMPG2, KIAA1549, KIZ, LRAT, MAK, MERTK, MVK, NEK2, NEUROD1, NR2E3, NRL, PDE6A, PDE6B, PDE6G, POMGNT1, PRCD, PROM1, RBP3, REEP6, RGR, RHO, RLBP1, RP1, RP1L1, RPE65, SAG, SAMD11, SLC7A14, SPATA7, TRNT1, TTC8, TULP1, USH2A, ZNF408, ZNF513, OFD1, RP2, RPGR, ABCC6, AFG3L2, ATXN7, COL11A1, COL2A1, JAG1, KCNJ13, KIF11, MFN2, OPA3, PAX2, TREX1, VCAN, ABCC6, ABHD12, ACBD5, ACO2, ADAMTS18, ADIPOR1, AFG3L2, AHI1, ALMS1, CC2D2A, CEP164, CEP290, CLN3, COL9A1, CSPP1, ELOVL4, EXOSC2, FLVCR1, GNPTG, HARS, HGSNAT, HMX1, IFT140, IFT81, INPP5E, INVS, IQCB1, LAMA1, LRP5, MKS1, MTTP, NPHP1, NPHP3, NPHP4, OPA3, PANK2, PCYT1A, PEX1, PEX2, PEX7, PHYH, PLK4, PNPLA6, P005, POC1B, PRPS1, RDH11, RPGRIP1L, SDCCAG8, SLC25A46, TMEM216, TMEM237, TRNT1, TTPA, TUB, TUBGCP4, TUBGCP6, WDPCP, WDR19, WFS1, ZNF423, OFD1, TIMM8A, ABHD12, ADGRV1, ARSG, CDH23, CEP250, CEP78, CIB2, CLRN1, DFNB31, ESPN, HARS, MYO7A, PCDH15, USH1C, USH1G, USH2A, BEST1, CAPN5, CRB1, ELOVL1, FZD4, ITM2B, LRP5, MAPKAPK3, MIR204, OPN1SW, RB1, RCBTB1, TSPAN12, ZNF408, ASRGL1, BEST1, C12orf65, CDH3, CNGA3, CNGB3, CNNM4, CYP4V2, LRP5, MFRP, MVK, NBAS, NR2E3, OAT, PLA2G5, PROM1, RBP4, RCBTB1, RGS9, RGS9BP, RLBP1, KSS, LHON, MT-ATP6, MT-TH, MT-TL1, MT-TP, MT-TS2, CACNA1F, CHM, DMD, NDP, OPN1LW, OPN1MW, PGK1, RS1, ABCA4, ARMS2, C2, C3, CFB, CFH, ERCC6, FBLN5, HMCN1, HTRA1, RAX2, TLR3, TLR4, or a combination thereof.

10. The method of claim 1, wherein the method results in regeneration of cells in the retina selected from the group consisting of retinal ganglion cells, photoreceptor cells, amacrine, bipolar cells, Müllner cells, horizontal cells, and a combination thereof.

11. The method of claim 1, wherein the injury is from hazardous radiation, physical force or intrusion, a retinal tear, retinal detachment, damaged induced by chemical agent(s), damage induced by neurotoxic agent(s), or a combination thereof.

12. The method of claim 1, wherein the neural degenerative disease is a disease of the central nervous system, said disease selected from the group consisting of Alzheimer's disease, amyotrophic lateral sclerosis, Friedreich's ataxia, Huntington's disease, Lewy body disease, Parkinson's disease, Spinal muscular atrophy, and a combination thereof.

13. A method of inducing regeneration of neuronal cells in a mammalian individual, comprising the step of providing to the individual a therapeutically effective amount of one or more Histone Acetyltransferase inhibitors (HATi).

14. The method of claim 13, wherein the individual has retinal injury and/or degenerative retinal neurons.

15. The method of claim 13, wherein the Histone Acetylation Transferase is from the GNAT family, MYST family, p300/CBP family, or is Rtt109.

16. The method of claim 13, wherein the HATi binds the active site of the histone acetylation transferase.

17. The method of claim 13, wherein the HATi does not bind the active site of the histone acetylation transferase.

18. The method of claim 13, wherein the HATi is SYC1127, A-485, C646, Garcinol, Anacardic acid, CPTH2, Curcumin, MB-3, Lys-CoA, H3-CoA-20, glycosaminoglycans, thiazinesulfonamide, isothiazolones, TH1634, ICG-001, benzylidenebarbituric acid, N1-aryl-propane-1,3-diamine derivative, cyclic peptide bromodomain derivatives, ischemin, N-phenyl (PU139) and N-benzyl (PU141) pyridoisothiazolones, tannic acid, BRD4, a functionally active derivative thereof, or a combination thereof.

19. A method of treating hearing loss, spinal cord injury, brain injury due to trauma, shock, or a combination thereof, comprising the step of providing to the individual a therapeutically effective amount of one or more Histone Acetylation Transferase inhibitors (HATi).

20. The method of claim 19, wherein the Histone Acetylation Transferase is from the GNAT family, MYST family, p300/CBP family, or is Rtt109.

21. The method of claim 19, wherein the HATi binds the active site of the histone acetylation transferase.

22. The method of claim 19, wherein the HATi does not bind the active site of the histone acetylation transferase.

23. The method of claim 19, wherein the HATi is SYC1127, A-485, C646, Garcinol, Anacardic acid, CPTH2, Curcumin, MB-3, Lys-CoA, H3-CoA-20, glycosaminoglycans, thiazinesulfonamide, isothiazolones, TH1634, ICG-001, benzylidenebarbituric acid, N1-aryl-propane-1,3-diamine derivative, cyclic peptide bromodomain derivatives, ischemin, N-phenyl (PU139) and N-benzyl (PU141) pyridoisothiazolones, tannic acid, BRD4, a functionally active derivative thereof, or a combination thereof.

24. A method of reducing the risk of retinal injury or disease, delaying the onset of retinal injury or disease, or reducing the severity of retinal injury or disease in an individual, comprising the step of providing to the individual a therapeutically effective amount of one or more Histone Acetylation Transferase inhibitors (HATi).

25. The method of claim 24, wherein the individual has diabetes, cardiovascular disease, high blood pressure, high blood sugar, being a smoker, excessive ultraviolet light exposure; high myopia, a personal history, and/or a family history.

26. The method of claim 24, wherein the HATi is SYC1127, A-485, C646, Garcinol, Anacardic acid, CPTH2, Curcumin, MB-3, Lys-CoA, H3-CoA-20, glycosaminoglycans, thiazinesulfonamide, isothiazolones, TH1634, ICG-001, benzylidenebarbituric acid, N1-aryl-propane-1,3-diamine derivative, cyclic peptide bromodomain derivatives, ischemin, N-phenyl (PU139) and N-benzyl (PU141) pyridoisothiazolones, tannic acid, BRD4, a functionally active derivative thereof, or a combination thereof.
